(19) United States
(12) Patent Application Publication
(10) Pub. No.: US 2016/0180694 A1
(43) Pub. Date: Jun. 23, 2016

(54) INFECTIOUS DISEASE WARNING SYSTEM WITH SECURITY AND ACCOUNTABILITY FEATURES

(71) Applicant: Mitchell ROSENBERG, Cherry Hill, NJ (US)

(72) Inventor: Mitchell ROSENBERG, Cherry Hill, NJ (US)

(21) Appl. No.: 14/908,985

(22) PCT Filed: Aug. 1, 2013
(86) PCT No.: PCT/US13/53273

§ 371 Date: Jan. 29, 2016

(51) Int. Cl. G08B 21/24; A61B 5/1171; A61B 5/1172

(52) U.S. Cl. CPC G08B 21/245; A61B 5/1172; A61B 5/1176

(57) ABSTRACT

A device adapted to be located at the entry of a patient room in an institution which detects a person approaching entry to or exit from the room and emits a simulated voice warning of any disease, medical condition, or type of isolation selected by an authorized person for the room, including an identification module which in some embodiments has one or more cameras, an identification badge reader, and/or a facial recognition module. The identification module may record persons entering as well as exiting a patient room. The device may also include means to determine compliance with apparel requirements for the room and may include means to simulate voice warnings such as “wash hands,” “wear gown,” “wear gloves,” “wear mask,” “dispose of gown,” and/or “wash hands after removing protective clothing.”

### CROSS-REFERENCE TO RELATED PATENT

This invention is related to an earlier invention of the same inventor as described in U.S. Pat. No. 8,421,623, Apr. 16, 2013, which is hereby incorporated by reference.

### BACKGROUND OF THE INVENTION

The present invention relates to the field of medical devices, more particularly to the field of contact isolation and infectious disease warning systems, security, and accountability.

The problem of preventing spread of infections within institutions such as hospitals, nursing homes, long term care facilities, and the like is very serious.

Patients who are contagious must be isolated and certain established protocols must be followed by institutional staff, physicians, and visitors.

When medically compromised patients occupy a hospital room, protocols are established which depend on the patient condition and must be taken to prevent transmitting disease to the patient or from the patient.

Warning visitors and staff of the precautions which must be taken regarding an individual patient under isolation has been a problem for hospitals. Due to the fact that hospitals are very busy places with many types of passive signs and other devices used for many purposes, it is not a rare occurrence for handwritten or even printed signs regarding infectious or other issues to be ignored or accidentally overlooked. Furthermore, placement of the sign can be ambiguous if the doors or entryways are adjacent with little space between them. In some cases signs are obscured by other signs, lab coats are hung obscuring the sign, the sign falls off or is obscured by objects or people. Also a paper or cardboard sign can be missed if a person approaches the room from the wrong angle or if the sign is dislodged.

In my prior patent I described a better, more active, yet inexpensive, solution to this problem.

However, my prior system and method did not provide means and steps to determine the identity of persons entering and leaving a patient's room or the date and time the person entered or left the room, nor did it provide means and steps to determine whether persons entering and leaving a patient's room have complied with dress and precaution protocols applicable to a particular patient's condition.

I have discovered that there is a need not only to warn persons entering a patient's room of any precautions required but also to identify the person entering a room subject to precaution protocols and to monitor and record compliance with such precaution protocols.

It is therefore an object of this invention to provide such a system and method to address the need for identifying persons entering and leaving a patient's room and also in some cases to determine whether such persons are complying or have complied with applicable dress protocols.

### SUMMARY OF THE INVENTION

These objects, and others which will become apparent from the following disclosure and accompanying drawings, are achieved by the present invention which comprises in one aspect a device adapted to be located at the entry of a patient room in an institution, the device comprising: a processor, memory, a voice generation system, and a set of programming instructions stored in the memory or processor, the programming instructions functioning to allow an authorized user to select one or more pre-programmed simulated voice warnings or one or more diseases, medical conditions, or types of isolation which corresponds to a pre-programmed simulated voice warning; one or more detectors adapted to detect a person approaching the entry of the patient room or to detect a person leaving a patient room; and an identification module, the identification module adapted to identify a person entering and, optionally, leaving the patient room, and optionally record the date and time the person enters and/or leaves the patient room; the device adapted to emit the selected simulated voice warning when entry of the room where the device is located is detected by the room entry detector, the one or more simulated voice warnings corresponding to the selected disease, medical condition, or type of isolation selected by the authorized user; and to identify a person entering, and/or leaving the patient room. In some embodiments, the voice warnings can be segregated so that the instructions to be followed before entering the room will be announced at detection of approach or entry and the instructions to be followed after leaving the room can be announced when an individual is detected leaving the room.

In another aspect the invention comprises a method of warning or notifying a person entering a patient's room of any infectious disease precautions required prior to entering the room and identifying the person comprising (A) providing such a device, (B) selecting by an authorized person one or more diseases, medical conditions, or types of isolation associated with a room, (C) detecting by the system a person approaching entry, (D) emitting a voice warning, (E) identifying the person by means of the camera or identification badge and, optionally, (F) recording the date and time the person enters and/or leaves the room.

In some embodiments the identification module comprises one or more cameras, an identification badge reader, and a facial recognition module.

In some embodiments the system and method not only identify the person but also automatically calculate the presence or absence of apparel worn by the person entering or leaving the room, the apparel being, for example, a mask, hat, protective clothing, gloves, scrubs, and the like. Some embodiments of the system automatically calculate whether or not the detected apparel complies with a preset apparel requirement protocol.

In some embodiments the system emits a voice or electronic display warning or both if incorrect apparel for the infection state of the patient is being worn by a person entering the patient room. In some embodiments when incorrect apparel is detected, an automatic notification is transmitted to a remote location such as a nursing station or administration of the institution.

In some embodiments the camera records video starting when a person is detected as entering the patient's room until the person is no longer within the view of the camera. The video can be stored in temporary memory and automatically retained for a predetermined period to allow for review if necessary. In some embodiments, the camera is enabled to continuously monitor the environment and, when motion is detected, to begin permanently recording into its memory beginning from a pre-determined amount of time prior to the actual motion detection and for a pre-specified period of time after motion is detected.

In certain embodiments the device includes biometric identification means to identify the person entering and/or leaving the patient room. Examples of such biometric identification means include facial, fingerprint, and iris recognition apparatus and associated algorithms. The identification module preferably includes a date and time recorder and in some embodiments is in communication with a remote device such as a nursing console or central control station so that the identity of each person entering or leaving a patient room is visible at the remote device. In some embodiments the remote device includes memory and programming to determine and record which persons enter which rooms at which times and to analyze behaviors and trends of individuals; for example if one individual repeatedly violates isolation protocols, disciplinary policies may be imposed.

Examples of voice warnings are “wash hands,” “wear gown,” “wear gloves,” “wear mask,” “dispose of gown,” “use hand sanitizer,” “wash hands with soap and water after removing protective clothing,” and combinations of said voice warnings. Examples of additional voice warnings which can be programmed into the system are listed in Table 1.

The system can comprise a portable unit which can be temporarily placed outside a patient room, for example on a door, door jamb, lintel, or wall near the entrance to the room. If preferred, the device can be permanently affixed in place or held in place by a permanent or temporary fixture.

The invention is an improvement over existing systems since it is much more unlikely that a visitor to a room housing a patient with a contagious medical condition or an immune-depressed medical condition will unwittingly enter the room without proper precautions if the visitor is warned by an alarm system which emits voice warnings, especially if the person is aware that a personal identification system and/or an apparel compliance detection system is in place. Therefore the invention improves compliance in at least two ways: by warning visitors and staff that a personal identification system is in place and thereby deterring non-compliance; and by actually identifying persons who do not comply with isolation protocols.

### BRIEF DESCRIPTION OF THE DRAWINGS

The description set forth above, as well as other objects, features and advantages of the present invention, will be more fully appreciated by referring to the detailed description and the drawing that follows.

FIG. 1 is a front perspective view of a device according to the invention, showing a touch screen with a welcome message, a speaker, a built-in camera, and an optional identification card or badge reader.

### DETAILED DESCRIPTION OF THE PREFERRED EMBODIMENTS OF THE INVENTION

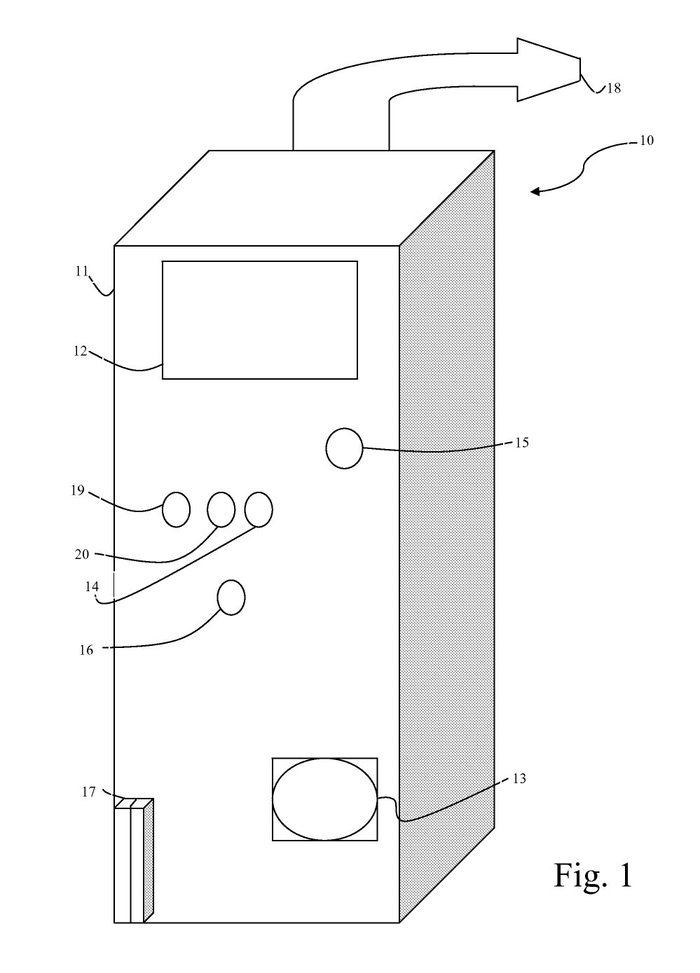

Reference is now made generally to FIG. 1 wherein one embodiment of a system according to the invention is illustrated, the system 10 having a box 11 having a touch screen 12 which displays a welcome screen having three selection modes, “select by disease entity,” “select by type of isolation,” and “select by individual voice warnings.” The box 11 includes a processor 19, memory 20, a sound generator 14 which is capable of generating voice simulations, and a speaker 13 as well as the touch screen 12. The box 11 also includes a motion detector or proximity detector 15. The motion or proximity detector 15 can be adjusted with respect to sensitivity and other factors using the touch screen 12 in setting mode, for example. The motion or proximity detector 15 can be a smart detector so that it can distinguish between irrelevant events such as a person passing by a room and a person actually entering the patient room. A camera 16 is included within box 11. An identification badge reader 17, which is optional, is also shown. The identification badge reader 17 may be the traditional type wherein the person swipes the badge or holds the badge near the reader or it may be an advanced type which does not require the person wearing the badge to take any step other than to approach within a few feet of the reader in which case the reader automatically detects the identity built into the card and processes the identity to calculate the identity of the person entering or leaving the room. The badge reader 17 can also be of the type that requires the individual to activate his badge in some way in order to be recognized.

The camera 16 can be directed in a forward direction to capture an image or video of a person detected as approaching the patient room or it can be directed both in both forward and reverse directions, or a second camera 18 can be directed into the patient room to capture an image or video of a person detected as leaving the patient room. A camera can also be located inside or outside the door of the patient room, for example a camera can be located 3-5 feet inside or outside the door. In the latter case a second proximity detector may be required. It is possible to provide a camera on the side, bottom of, or adjacent to the device so that it can detect both approaches and departures from a room.

When a person is detected approaching or leaving a room, reference may be made to an internal clock to enable recording of the date and time the person is detected. At the same time, the person may be identified by biometric recognition such as fingerprint, facial recognition, iris recognition, and the like. In some embodiments the system detects whether a person is approaching a patient room or is leaving the room and records data of such approach or departure.

In some embodiments the camera or other means can be used to acquire digital images which are then processed by the system according to an algorithm to automatically calculate the presence or absence of apparel worn by the person entering or leaving the room, the apparel being, for example, a mask, hat, protective clothing, gloves, and the like, and in some embodiments the data is processed to calculate whether or not the detected apparel complies with a preset apparel requirement protocol. Various apparel protocol combinations are set forth in the following tables which refer to various apparel requirements.

In certain embodiments the presence of apparel can be detected by other than camera means, for example an RFID may be embedded in each mask, gown, hat, etc., and identified by an RFID reader which can detect presence or absence of each item on a person entering or leaving a patient room.

In some embodiments the apparatus and method allow an institution's administration to identify offenders of isolation protocols by reviewing images and/or video either in real time or by playing back video or images stored in the memory.

Table 1 lists a more comprehensive list of possible voice warnings which can be pre-programmed in certain other embodiments of the invention.

TABLE 1
List of Voice Warnings

Wash hands before entering
Wash hands before contact
Wash hands before putting on gloves
Wash hands after contact
Wash hands before and after contact
Wash hands after removing gloves
Wash hands after leaving the room
Wash hands with soap and water
Wash hands with soap and water after removing gloves
The use of gowns, gloves and eye protection is based on the task to be
performed and the risk of exposure to blood and/or body fluids
Gowns, gloves and mask are to be worn for all patient contact and to be
donned before entering the room
Gowns and gloves are required for all entering
Gowns and gloves are required for all patient contact
Gowns and gloves are required for all environmental contact
Dispose of gown in room
Dispose of gloves in room
Dispose of gown after leaving room
Do not enter this room unless you are immune to Chicken Pox
Do not enter unless you are immune to Rubella also known as
German Measles
Do not enter this room unless you are immune to Rubeola
See nurse before entering this room
Susceptible persons may not enter this room
This is a negative pressure room
Keep door closed
Wear eye protection if splashing is likely
Wear mask
Wear mask if within 3 feet of patient
Wear N-95 mask or powered air respirator
Wear fitted TB mask or powered air respirator
Surgical masks and gloves required for all entering
Wear gown if contact with secretions possible
Do not enter if you have an active infection such as a sore throat, fever,
diarrhea etc.,
No children under 14 allowed
No fresh flowers or plants permitted
Wear gown if contamination of visitor or caregiver is anticipated
Use dedicated patient equipment
Do not enter, see nurse

In Table 2, an example is shown of a set of simulated voice warning announcements which are automatically made by the device when certain medical conditions are selected by the nurse or other professional staff and the motion detector detects a person entering the room.

TABLE 2
Voice Warnings By Medical Condition

| Medical Condition | Synthetic Voice Warning |
|---|---|
| MRSA | Gowns and gloves are required for all entering, wash hands before and |
| | after contact, wash hands after removing gloves, dispose of gown and |
| | gloves in room, wear eye protection if splashing is likely, use dedicated |
| | patient equipment |
| C. diff | Gowns and gloves are required for all entering, wash hands before and |
| | after contact, wash hands with soap and water after removing gloves, |
| | dispose of gown and gloves in room, wear eye protection if splashing is |
| | likely, use dedicated patient equipment |
| Vancomycin- | Gowns and gloves are required for all entering, wash hands before and |
| resistant | after contact, wash hands after removing gloves, dispose of gown and |
| Enterococcus | gloves in room, wear eye protection if splashing is likely |
| Klebsiella | Gowns and gloves for all entering, wash hands before and after contact, |
| pneumonia | wash hands after removing gloves, dispose of gown and gloves in room, |
| | wear eye protection if splashing is likely, use dedicated patient equipment |
| H1N1 Influenza | Wear mask if within 3 feet of patient, wear N-95 mask or powered air |
| | respirator, gowns and gloves are required for all entering |
| Tuberculosis | This is a negative pressure room, wash hands before entering the room and |
| | after removing gloves, wear fitted TB mask or powered air respirator, wear |
| | gloves if contact possible with blood or body fluids, gowns are required if |
| | contamination of clothing is possible either by blood or body fluids or a |
| | contaminated environment, keep door closed. |
| Shingles, Zoster | Gowns and gloves for all entering, wash hands before and after contact, |
| | wash hands after removing gloves, dispose of gown and gloves in room, |
| | wear eye protection if splashing is likely, do not enter this room unless you |
| | are immune to Chicken Pox |

Upon selection of a medical condition, e.g., C. diff. or MRSA, the system automatically determines the selection of appropriate voice warnings but would not announce or display the disease or medical condition, e.g., C. diff., due to privacy concerns. The device could continuously display a visual warning such as “Contact Isolation” or “Droplet Precautions” or an instruction such as “Wash Hands Before Entering” or a safety concern such as “Fall Risk” until the device is activated by an individual approaching or entering the room. Programming and associated methods which enable user selection of a medical condition only, after which the device would determine the appropriate warnings to be announced, may be preferred in some situations, limiting the complexity of programming options.

In some embodiments an authorized user such as a nurse has an option to select a type of isolation, in which case the pre-programmed voice warning corresponding to that type of isolation are emitted by the device upon detection of a person entering the room of the patient. Table 3 lists some examples of possible voice warnings corresponding to such types of isolation.

TABLE 3
Voice Warnings by Type of Isolation

| Type of Isolation | Voice Warning |
|---|---|
| Standard Precautions | wash hands before and after contact; the use of gowns, |
| | gloves and eye protection is based on the task to be |
| | performed and the risk of exposure to blood and/or body |
| | fluids. |
| Contact Precautions | Wear gowns, wear gloves, wash hands before and after |
| | removing gloves, wear mask if splashing of body fluids is |
| | possible |
| Airborne Precautions | This is a negative pressure room, wash hands before |
| | entering the room and after removing gloves, wear mask, |
| | wear gloves if contact possible with blood or body fluids, |
| | gowns are required if contamination of clothing is |
| | possible either by blood or body fluids or a contaminated |
| | environment, keep door closed. |
| Droplet Precautions | Surgical masks and gloves required for all entering, keep |
| | door closed, wear gown if contact with secretions |
| | possible, wash hands before and after patient contact, keep |
| | door closed. |
| Neutropenic Precautions | Do not enter if you have an active infection such as a sore |
| | throat, fever, diarrhea etc., no children under 14 allowed, |
| | wear gloves, wash hands before and after contact, no fresh |
| | flowers or plants permitted, wear gown if contamination |
| | of visitor or care giver is anticipated |

The device of the invention can be self-contained or can be part of another device or system. The system can include one device or, preferably, will include many devices located at a plurality of rooms in an institution. The programming can take place at the device, for example by a nurse selecting warnings as one would select from a menu of options on a dishwasher, washing machine, or dryer, or can take place at a nursing console or even remotely at a hospital administrator computer which oversees many rooms or with an authorized smart phone using an application (“app”) or over a simple internet connection. An authorized person at a remote location can communicate with the device to make selections from the menus and receive data regarding identity of persons entering or leaving the room and the time the persons enter or leave, including image and video information, by any of a variety of communications means and protocols, for example Bluetooth, wi-fi, radio frequency, hard wire, smart phone app, or integration with the institution's computer information systems. An example of a current institutional computer system is the Computerized Physician Order Entry (CPOE) and Electronic Medical Record (EMR) system. Hospitals equipped with CPOE and EMR use very little paper. In some embodiments the device can be integrated with an institution's CPOE/EMR or other centralized computer system so that a physician, nurse, or other authorized person may order isolation via the centralized computer system which would immediately trigger the device to activate. Such activation could occur during admission or during the course of a hospital stay or stay in a different type of institution. The communication would automatically engage the device which would then begin to function immediately as a motion detector with the appropriate warnings and image and/or video identification of person's entering the room where the activated device is located. Such CPOE systems can be smart systems which recognize certain terms in a diagnosis of a patient or in a lab report and then automatically send instructions to the device at that patient's room so that appropriate warnings can be automatically programmed. Such systems in some cases can obviate the need for a nurse at a nursing station to make selections from the menus associated with the device.

The set of instructions which constitute the programming can be in C++ or any of a variety of programming languages, and would normally be programmed into the processor. The simulated voice messages can be recorded by a person or can be synthesized from a dictionary of words or sounds.

A manual or remote override can be provided which allows an authorized person such as a nurse or other medical staff visitor to the room to suppress the voice warning on a one time or on an as needed basis if it becomes annoying. In some embodiments the system can be programmed to suppress or lower the volume on any audio warning during certain hours, for example during hospital “quiet periods,” in which case only visual warnings are implemented. Visual warnings can include, for example, flashing information such as “CONTACT ISOLATION” on a system display. The system clock can be used to implement blackout periods or quiet time periods. During quiet periods the proximity detector, motion detectors, cameras and identification module will not be affected and will operate as during normal periods.

The device can be temporarily placed on a door jamb, door, door frame, lintel or wall at or near the entrance of a patient room in some embodiments. The device can also be permanently placed on or in a door jamb, door, door frame, lintel or wall. The device will also include in most embodiments a motion detector 15 for detecting the approach of a visitor to the patient's room as well as individuals leaving the patient's room. This can be accomplished using one or more motion detectors. The motion detector is preferably focused so that it has a narrow field of view, for example a 15 degree field of view, and can be a passive infrared (PIR) or ultrasound type, set to detect a person at a pre-specified distance, for example at 3 to 5 feet, or a laser or IR beam sensor of conventional or specialized type. The motion detector 15 can also be of an advanced type utilizing the camera function wherein data from the camera can be calculated and, using an algorithm, approach by a human can be detected based on information captured by the camera. The device may be placed at a location and configured such that the motion detector would not activate the alarm if a person is passing by but not entering the room, or may include hardware and software to determine whether a person detected by the motion detector is the patient or a nurse for whom the warning is not necessary or is undesirable. The motion detector function of the device may be adjustable so that higher or lower sensitivity may be selected so that the alarm functions when people are entering the room but not when they are passing by the room so that in embodiments comprising a camera means of identification only visitors and staff actually entering a hospital room are photographed.

The device can be programmed to allow selection of one or more languages, for example in certain institutions both English and Spanish language announcements should be made whereas in other institutions announcements in both English and French or another commonly used language should be made. The device may be adapted to use conventional alternating current or may use battery power, in which case a low battery warning module can be included. An on/off switch or button can also be included in certain embodiments.

The device can also include, in some embodiments, a touch pad and programming to request a password and to indicate successful activation.

The device could continuously display a visual warning such as “Contact Isolation” or “Droplet Precautions” or an instruction such as “Wash Hands Before Entering” or a safety concern such as “Fall Risk” until the device is activated by an individual approaching or entering the room. The device may also include a visual warning such as flashing lights or a flashing sign such as “contact isolation, see nurse” which will flash when a visitor is detected entering the room.

The device may include a selection option on a touchpad to toggle between “select voice warning message,” “select disease or medical condition,” and “select type of isolation” modes.

In some embodiments selection by an authorized user of safety-type information such as “patient is blind,” “patient is fall risk,” “do not draw blood from left arm,” and the like can be enabled so that audio warnings for such selections can be generated upon approach by a person to a patient room where such safety-type condition is applicable.

The system and method of the invention are well adapted to solve a serious problem in hospitals and other institutions where patients may have diseases which require special precautions to protect those entering the patient's room as well as to protect the patient and to prevent transmission of disease between them, or between them and other parties, wherein the state of the art is to post a sign indicating the precautions to be taken, wherein such signs are often unnoticed, and wherein there is no electronic means to identify persons entering and leaving such rooms where precaution protocols are in place. This invention provides an easily and quickly programmable system which allows selection of a disease or condition or type of isolation by an authorized person, wherein the selection of disease is not announced to those entering the room and thus complying with privacy requirements, and wherein a voice announcement of precautions to take is calculated from the disease, medical condition, or type of isolation, wherein such voice announcement is automatically made when the system detects a person entering the patient room and wherein either the identity of a person entering a patient's room is be detected or wherein compliance with apparel protocol applicable to a particular room is detected.

Though the invention has been described with respect to a number of embodiments, many additional variations and modifications will immediately become apparent to those skilled in the art. It is therefore the intention that the appended claims be interpreted as broadly as possible in view of the prior art to include all such variations and modifications.

1. A system comprising a device adapted to be located at the entry of a patient room in an institution, the device comprising: (a) a processor, (b) memory, (c) a voice generation system, (d) a set of programming instructions stored in the memory or processor, the programming instructions functioning to allow an authorized user to select one or more pre-programmed simulated voice warnings and/or one or more diseases, medical conditions, or types of isolation which corresponds to a pre-programmed simulated voice warning; (e) one or more detectors adapted to detect a person approaching the entry of the patient room or to detect a person leaving a patient room; and (f) an identification module, the identification module adapted to identify a person entering and/or leaving the patient room; the device adapted to emit the selected simulated voice warning(s) when entry of the room where the device is located is detected by the one or more room entry detectors, the one or more simulated voice warnings corresponding to the selected disease, medical condition, or type of isolation selected by the authorized user; and to identify a person entering and/or leaving the patient room.

2. The system of claim 1 wherein the identification module (f) comprises one or more members selected from the group consisting of one or more cameras, an identification badge reader, a fingerprint identification module, an iris recognition module, and a facial recognition module.

3. The system of claim 1 programmed to record the date and time a person is detected entering and/or leaving a patient room.

4. The system of claim 1 wherein the simulated voice warnings are selected from the group consisting of “wash hands,” “wear gown,” “wear gloves,” “wear mask,” “dispose of gown,” “wash hands after removing protective clothing,” and combinations of said simulated voice warnings.

5. A system of comprising a plurality of the devices adapted to be located at the entry of a patient room in an institution of claim 1 and a remote device in communication with each of the plurality of devices adapted to be located at the entry of a patient room in an institution, the remote device adapted to remotely select one or more simulated voice warnings to be emitted for each of the plurality of devices; wherein the remote device is configured to allow the authorized user to alternatively select one or more diseases and/or medical conditions or types of isolation from one or more menus, each selection corresponding to one or more of the pre-programmed simulated voice warnings; and wherein the identification module is programmed to communicate the identity of a visitor or staff member entering the patient room to the remote device.

6. The system of claim 1 further comprising means to remotely select one or more diseases and/or voice warnings to be emitted upon approach by a person to the room or upon exit by a person from a room.

7. (canceled)

8. The system of claim 1 wherein the device comprises a (g) touch pad and programming to request a password, display a plurality of selectable medical conditions, and to indicate successful activation.

9. (canceled)

10. The system of claim 5 wherein the plurality of devices each further includes (i) one or more motion detector modules for determining when a person is entering and/or leaving a patient room.

11. (canceled)

12. A method of warning visitors and medical staff members of precautions necessary prior to entering a patient room in an institution comprising (A) providing a system according to claim 1, (B) selecting by an authorized person one or more diseases, medical conditions, or types of isolation associated with a room, (C) detecting by the system a person approaching entry, (D) emitting a voice warning, and (E) identifying the person by means of the identification module.

13. The method of claim 12 wherein the identification module comprises one or more cameras and/or one or more identification badge readers.

14. The method of claim 12 further including (F) recording the date and time an identified person enters and/or leaves the room.

15. (canceled)

16. (canceled)

17. The method of claim 12 further including (I) determining by means of the camera whether a person entering the room is wearing apparel required for persons entering the room and, if incorrect apparel is determined, emitting an audio or visual signal indicating incorrect or insufficient apparel being worn by the person, the sufficiency of the attire apparel calculated according to a programmed correspondence table.

18. (canceled)

19. The method of claim 12 comprising detecting the attire of the visitor or staff member entering and/or leaving the patient room, the attire selected from the group consisting of gloves, head covering, mask, shoe covering, gown, jacket, scrubs, and combinations thereof.

20. The method of claim 12 further comprising detecting the identity of a person entering and, optionally, leaving a patient room in an institution by face recognition and/or by detecting identity from an identification card or device carried by the person.

21. The method of claim 12 comprising recording a photograph and/or video of a person entering and, optionally, leaving the patient room.

22. The method of claim 12 wherein the device is programmed to record video for a preselected time upon detection of a person approaching the patient room and, optionally, to record activity within the room.

23. The method of claim 12 comprising providing unique identifiers on each item of apparel provided to persons and providing electronic detection means to determine presence or absence of an item of apparel worn by a person detected as entering or leaving a patient room.

24. The method of claim 12 further comprising identifying the person at a remote device by means of the identification module.

25. The method of claim 24 wherein the remote device is a nursing console.
